(19) United States
(12) Patent Application Publication
(10) Pub. No.: US 2016/0089409 A1
(43) Pub. Date: Mar. 31, 2016

(54) A METHOD AND COMPOSITION USING PARSLEY (PETROSELINUM CRISPUM) AS A NATURAL REMEDY FOR DIABETES MELLITUS

(71) Applicant: UMM AL-QURA UNIVERSITY, Makkah (SA)

(72) Inventor: Saeed Saeed Alghamdi, Makkah (SA)

(73) Assignee: UMM AL-QURA UNIVERSITY, Makkah (SA)

(21) Appl. No.: 14/417,636

(22) PCT Filed: Sep. 30, 2014
(86) PCT No.: PCT/IB2014/001967

§ 371 Date: Jan. 27, 2015

(51) Int. Cl. A61K 36/23; A61K 36/899; A61K 36/324; A61K 36/71

(52) U.S. Cl. CPC A61K 36/23; A61K 36/71; A61K 36/899; A61K 36/324

(57) ABSTRACT

A homogenate to be administered to a patient to inhibit and reduce increased glucose level in the blood and stimulate insulin secretion. The homogenate includes the use of different components, mainly the herb Petroselinum Crispum (parsley) to be administered to a patient at a particular dosage for a predetermined period of time depending on the severity of the case, and that effectively decreases and controls the patient's glucose levels and insulin secretion.

### BACKGROUND OF THE DISCLOSURE

1. Technical Field

The present disclosure relates to a composition comprising the herb Petroselinum Crispum together with one or more other natural components and/or an active ingredient derived therefrom and a method for administering the composition to a patient to inhibit and/or treat harmful effects of diabetes mellitus.

2. Description of the Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present disclosure.

The current disclosure describes the use of a combination of herbs, seeds, roots and flowers of different plants to treat diabetes mellitus. According to the American Heart Association, the risk of stroke is two to four times higher for people with diabetes, and the death rate from heart disease is two to four times higher for people with diabetes than for people without the disease.

Plants started being used as medicine as early as 3000 BC. Herbal medicine has a long history of being successful in healing and improving different conditions. Different cultures started using herbs at different parts of the world around the same time, it was later found that people used the same or similar herbs to treat similar conditions, and with the development of science, research and analysis, extracts and active ingredients of different herbs began to be used to form medical drugs.

Parsley, Petroselinum Crispum is a species of Petroselinum in the family Apiaceae, and is native to the central Mediterranean region. It is widely known as an herb/spice/vegetable. Parsley contains no cholesterol; however, it is a very rich herb in anti-oxidants, vitamins, minerals and dietary fiber; minerals like potassium, calcium, manganese, iron, and magnesium; and vitamins like as vitamin K, vitamin C, and vitamin A. Parsley contains health benefiting essential volatile oils that include myristicin, limonene, eugenol, and alpha-thujene.

Parsley is widely used to help improve various health conditions. The herb was used medicinally prior to being consumed as a food. It is thought to help lower risk of cancer, enhance the immune system, lower the risk of stroke, heart attack and thickening of artery walls and thus enhancing the performance of the heart. It is also known to help with urinary tract infections, treat inflammations and as having a very powerful detox effect.

Boswellia gum also known as the “Indian Frankincense”, is the resin/gum of the boswellia serrata tree which is a species in the family burseraceae and is native to India and tropical regions of Africa and Asia. The natural gum is ancient; it dates to thousands of years back. It has been widely used as a perfume, an incense and medicine. It is collected by purposefully and repeatedly wounding the boswellia seeata tree through its ash-colored papery bark into the sapwood; the tree then bleeds a waxy structured resin/gum that quickly hardens and becomes glossy. It is yellowish in color and darkens as it ages. It is rich in boswellic acids, having at least 50% and at most 75% boswellic acids but preferably having 65% of boswellic acid that is beneficial in preventing the formation of leukotriene in the body.

Nigella Sativa seed, comes from an annual herbaceous plant. The seed is commonly known as the black seed, black cumin or “Habbatul Barakah” in Arabic, which directly translates to the “seed of blessing”. It is from the family Ranunculaceae, and is native to the Middle East and Far East Asia and Europe regions. The black seed has long been used in folk medicine in these regions. It has a rich composition of proteins, minerals, carbohydrates and fats. Almost all of its fat content is in the form of omega-3 and omega-6 fatty acids, which are important for good overall health and a sound immune system. It also has around 15 amino acids, the building blocks of protein, 9 of which are the essential amino acids.

The black seed contains over 100 valuable nutrients. It has approximately 21% protein, 38% carbohydrates, and 35% plant fats and oils. Nigellone, thymoquinone and fixed oils are considered the active ingredients of nigella sativa seed. It also contains proportions of protein, carbohydrates and essential fatty acids. Other ingredients include linoleic acid, oleic acid, calcium, potassium, iron, zinc, magnesium, selenium, vitamin A, vitamin B, vitamin B2, niacin, and vitamin C.

Nigella sativa seed is very small yet very powerful, different historical stories and studies show that it has vital anti-inflammatory, anti-bacteria, and anti-fungal properties. It also reduces fasting blood sugar levels and decreases insulin resistance. The seed stimulates the regeneration of dying beta cells within diabetic pancreas, and helps with epilepsy, oral, brain, breast and colon cancers.

Wheat is a grain that was originally native to southwestern Asia. However, is currently being cultivated worldwide. It belongs to the family Poaceae. Wheat is rich in catalytic elements, mineral salts, calcium, magnesium, potassium, sulfur, chlorine, arsenic, silicon, manganese, zinc, iodide, copper, vitamin B, and vitamin E. It is greatly beneficial in helping with various diseases, such as coronary diseases, breast cancer. It also helps regulating obesity especially in women, prevents chronic inflammations and childhood asthmas.

Research has proved that due to its low fat content, wheat helps lower the hazards of heart diseases. It is also rich in the mineral magnesium, which acts as a co-factor to more than 300 enzymes that are involved in the body's use of insulin and glucose secretion, meaning wheat helps regulate blood glucose levels in diabetic patients.

Barley is a grain that belongs to the family of Plantae. It is native to eastern Asia and eastern Mediterranean. Barley has high nutritional values. It is rich in vitamin A, vitamin C, thiamin, choline, vitamin D, vitamin E, vitamin B12. It is also a great source of minerals; barley has calcium, iron, magnesium, potassium, sodium, zinc and copper.

A homogenate was created by using all the different previously described ingredients. The homogenate helps control the glucose levels in the blood of diabetic patients. The homogenate in a conventional dosage form has characteristics that are different from modern medicines, such as consistency, reproducible quality, safety, physical, chemical and microbiological stability and bioavailability.

### BRIEF SUMMARY OF THE DISCLOSURE

The foregoing paragraphs have been provided by way of general introduction, and are not intended to limit the scope of the following claims. The described embodiments, together with further advantages, will be best understood by reference to the following detailed description taken in conjunction with the accompanying drawings.

One embodiment of the disclosure relates to a method for administering a composition comprising parsley (Petroselinum Crispum), water, boswellia gum, nigella sativa seeds, wheat and barley to an individual in amounts effective to inhibit or decrease the glucose level in the blood of a human suffering from diabetes and stimulate insulin secretion.

Insulin is the protein responsible for facilitating glucose entry into cells. Insulin secretion is stimulated with high glucose level in the blood. A diabetic patient already has a high glucose level in the blood, and upon administering the homogenate the body is provided with nutrients that help further increase the glucose level in the blood and hence stimulate insulin secretion. A healthy human has a normal fasting glucose concentration that ranges from 80-90 mg/100 ml associated with low levels of insulin secretion. Administering the homogenate of this disclosure to a healthy human will increase the fasting glucose concentration to at least 120 mg/100 ml of blood causing the stimulation of insulin secretion.

In another embodiment the method comprises dissolving or suspending the homogenized ingredients in an aqueous composition to form a homogenized composition.

In another embodiment the method comprises administering the mixture to a patient suffering from diabetes four times a day. Each dosage is administered in an amount of at least 1.9 g/kg so that the total amount administered is 7.6 grams mixture per kg patient per day. Preferably each dosage is administered as a composition having total volume of about 250 ml, e.g., from 50 to 500 ml, preferably 100 to 400 ml or 200 to 300 ml. The same dosages may be used for obese patients regardless of age.

In another embodiment the method comprises administering the mixture to a patient four times a day in an amount of at least 0.95 g/kg if the patient is less than 12 years of age for a total daily dosage of 3.8 grams mixture per kg patient per day. Preferably each dosage is administered as a composition having total volume of about 125 ml, e.g., from 50 to 300 ml, preferably from 100 to 200 ml.

In another embodiment the homogenate prevents or treats the harmful effects of one or more medical conditions associated with type 1 diabetes including thyroid disorders, celiac disease and Addison's disease.

In another embodiment the homogenate prevents or treats the harmful effects of one or more medical conditions associated with type 2 diabetes including damage to the body, dehydration and diabetic coma.

In another embodiment the homogenate prevents or treats the harmful effects of one or more medical conditions associated with buildup of glucose in blood including heart and vessel diseases, nerve damage, kidney damage, eye damage, foot damage, skin and mouth conditions, osteoporosis, Alzheimer's and hearing problems.

In another embodiment the homogenate is administered orally in liquid form.

In another embodiment the homogenate in liquid form further includes syrup, artificial sweetening agents, or natural fruit flavoring agents.

In another embodiment the patient is not to administer the homogenate for a period that is longer than 30 consecutive days or less than 7 consecutive days.

### DETAILED DESCRIPTION OF THE EMBODIMENTS

The present disclosure relates to a homogenate that may be administered to a patient diagnosed with diabetes in an amount effective to prevent the damaging effects of increased glucose levels in the blood.

The homogenate includes components obtained from the herb Petroselinum Crispum (parsley), boswellia gum, nigella sativa seeds, wheat, barley and water. Any suitable variety and/or amounts of these components may be of use in the present disclosure but preferably the amount being used is 500 grams of Petroselinum Crispum (parsley), 1 liter of water and 10 grams of each of the other components and, in any case, an amount effective for inhibiting or treating the harmful effects of diabetes mellitus. In another embodiment, the homogenate consists of only Petroselinum Crispum (parsley), water, boswellia gum, nigella sativa seeds, wheat and barley materials. In still another embodiment the homogenate consists essentially of the Petroselinum Crispum (parsley), water, boswellia gum, nigella sativa seeds, wheat and barley materials and excludes synthetic or non-naturally derived pharmaceutical agents.

The homogenate may be prepared according to conventional techniques. In one embodiment 500 grams of clean/wet Petroselinum Crispum, parsley (leaves and trunks) is mixed with one liter of water for a period of time between 5 and 20 minutes but preferably the time is 15 minutes. Then, 10 grams of each of the boswellia gum, nigella sativa seeds, wheat and barley is added and the resulting mixture is mixed for a period of time between 1 and 5 minutes but preferably the time is 5 minutes.

In a preferable embodiment the homogenate is administered to a patient having diabetes mellitus 4 times a day in an amount of 1.9 g/kg of patient weight, preferably in an amount of at least 2.5 g/kg, 3 g/kg or 4 g/kg. The homogenate is preferably administered starting in the early morning with a time period of 4-12 hours, preferably 5-10 hours or 6 hours between every administration.

In one embodiment, the homogenate prevents harmful effects of one or more medical conditions associated with type 1 diabetes including thyroid disorders, celiac disease, and/or Addison's disease.

Thyroid disorders may be due to too much thyroid hormone production “hyperthyroidism” or too little thyroid hormone production “hypothyroidism” and both may be accompanied by an enlarged thyroid gland, a condition named “goiter”. Symptoms of hypothyroidism are not very apparent, and may be more obvious if the condition worsens. These symptoms include weight gain at regular food intake, depression or feeling sluggish, muscle weakness, dry skin, hair loss, poor memory, and difficulty concentrating. However, symptoms of hyperthyroidism are very obvious and may include increased perspiration, intolerance to heat, fatigue, difficulty sleeping, nervousness, irritation and a fast heartbeat.

Celiac disease is a disease that affects the intestine's ability to tolerate the protein “gluten” causing damage to the intestine's ability to absorb nutrients over time. This may result in mal-nutrition and that can lead to a variety of problems. Addison's disease is an autoimmune disease where the immune system attacks the body's healthy tissue. It involves not enough production of the hormones “Coristol” and “Aldosterone” of the adrenal glands. Those hormones help control a lot of the blood functions especially in the area of response to stress, these hormones also affect blood pressure, heart function, body's response to insulin, fluid balance and blood pressure. Symptoms of the Addison's disease may include but are not limited to fatigue, muscle weakness, appetite loss, or weight loss. Some people experience nausea, vomiting, diarrhea, dizziness, low blood pressure, and skin discoloration.

In another embodiment the homogenate prevents the harmful effects of one or more medical conditions associated with type 2 diabetes including damage to the body, dehydration and diabetic coma. Damage to the body occurs over time, where it can damage the nerves and small blood vessels of the eyes, kidneys and the heart. Damage to the heart's blood vessels may eventually result in atherosclerosis and hardening of arteries, which leads to heart attacks and increases the chances of strokes.

Dehydration is caused because of the increase in urination associated with high glucose levels in the blood. Diabetic coma is considered a life-threatening complication, but is only the case with the condition of the patient worsens and he is dehydrated and unable to make up for the fluid loss in their body.

In another embodiment the homogenate prevents the harmful effects of one or more medical conditions associated with buildup of glucose in blood including heart and vessel diseases, nerve damage, kidney damage, eye damage, foot damage, skin and mouth conditions, osteoporosis, Alzheimer's and hearing problems.

Different nerves in the human body control different functions. Damage to the capillaries that feed the nerves due to high glucose in the blood may lead to nerve mal-nutrition and thus mal-function. For diabetic patients this is especially a problem in the feet. Damaged nerves can cause tingling, numbness, burning or pain that begins at the tips of the toes and gradually spreads upward. Poorly controlled blood sugar can eventually cause loss of sensation to the affected limb.

While damaged small blood vessels (capillaries) may lead to nerve complications, it also is the reason behind kidney and eye damage. Kidneys use clusters of millions of tiny blood vessels to filter the blood; any damage may lead to kidney failure or serious kidney conditions.

Damaged tiny blood vessel function in the eye may damage the retina or lead to high eye blood pressure a condition called “glaucoma”.

The homogenized composition of Petroselinum Crispum, parsley can be administered to a patient to address one or more of the above-noted conditions or symptoms of diabetes mellitus. In one embodiment of the disclosure a homogenate that comprises processed Petroselinum Crispum (parsley) together with one or more of the previously described components is suitable for nutriceutical or pharmaceutical use by oral administration.

Liquid preparations are in the form of homogeneous aqueous suspensions or solutions using the whole Petroselinum Crispum, parsley (leaves and trunk). Such formulations have acceptable taste and mouth feel. The preparation of a fine and homogenous dispersion of processed Petroselinum Crispum, parsley is important to the liquid formulations.

Because of the uniformity and fineness of the dispersion, there is a greater degree of contact between the homogenate particles and the papillae on the tongue. Therefore, the homogenate dispersion of this disclosure exhibits “roundness” of taste particularly for a liquid product.

One embodiment of the disclosure includes the composition in liquid form. Thus, according to a first specific aspect of the disclosure, there is provided a liquid dosage formulation, in the form of a homogeneous dispersion in water of Petroselinum Crispum, parsley material in finely divided form.

In such formulations, the nutritional principles are better assimilated. The fineness and uniformity of the dispersion may also lead to significantly improved physical stability. The dispersion is physically stable over a long period and is not prone to separation. If some separation does occur, the homogenized product redisperses easily by simple shaking and reverts to its original homogeneous appearance. There is also a significant reduction or attenuation of the typical “collar” effect (separation and also floating of some suspended particles above the separated fluid, and sediment at the bottom part of the bottle) that is commonly observed with suspension formulations, such as extemporaneously prepared dispersions of fruit juices.

By “homogeneous” in this context is meant that the particles of Petroselinum Crispum (parsley) and the previously described components (water, boswellia gum, nigella sativa seeds, wheat and barley) present in the formulation are sufficiently fine and uniform that the formulation feels smooth in the mouth when presented in a liquid dose form. In quantitative terms, “homogeneous” may mean that all or substantially all of the Petroselinum Crispum (parsley), water, boswellia gum, nigella sativa seeds, wheat and barley materials are present in the form of particles having a particle size of less than 500 μm, and more preferably less than 100 μm. For instance, more than 90% w/w of the Petroselinum Crispum, parsley, water, boswellia gum, nigella sativa seeds, wheat and barley materials is preferably present in the form of particles with a size less than 100 μm, or a size less than 80 μm, or a size less than 50 μm. It may well be that the majority of the Petroselinum Crispum, parsley, water, boswellia gum, nigella sativa seeds, wheat and barley materials, e.g. more than 90% w/w or more than 95% w/w, is present in the suspension in the form of particles with a size in the range 1-50 μm.

In this aspect of the disclosure the amount of Petroselinum Crispum (parsley), on dry basis, will generally be at least 45% w/w, and up to 55% w/w, of the weight formulation, preferably between 45% w/w and 55% w/w, e.g. about 50% w/w of the total weight of all components except water. “Dry basis” in the context of the present disclosure means the total weight of the homogenate not including the water contributed by the aqueous solution. The weight of parsley is the weight when harvested. In another embodiment the homogenate comprises the parsley in an amount of from 100 g to 1 kg per one liter of water, more preferably the parsley is present in an amount of 200-900 g, 300-800 g, 400-700 g, 500-600 g per one liter of water.

The amount of boswellia gum, on dry basis, will generally be at least 0.4% w/w on a dry basis, and up to 10% w/w but preferably 1.4% w/w, of the weight formulation, preferably between 0.4% w/w and 1% w/w, e.g. about 0.5% w/w of the total weight of all components except water.

The amount of nigella sativa seeds, on dry basis, will generally be at least 0.4% w/w, and up to 10% w/w but preferably 1.4% w/w, of the weight formulation, preferably between 0.4% w/w and 1% w/w, e.g. about 0.5% w/w of the total weight of all components except water.

The amount of wheat, on dry basis, will generally be at least 0.4% w/w, and up to 10% w/w but preferably 1.4% w/w, of the weight formulation, preferably between 0.4% w/w and 1% w/w, e.g. about 0.5% w/w of the total weight of all components except water.

The amount of barley, on dry basis, will generally be at least 0.4% w/w, and up to 10% w/w but preferably 1.4% w/w, of the weight formulation, preferably between 0.4% w/w and 1% w/w, e.g. about 0.5% w/w of the total weight of all components except water.

The homogenate may be prepared by dispersing the components in water, and subjecting the dispersion to homogenization. Such a process represents a further aspect of the disclosure.

Homogenization of the Petroselinum Crispum, (parsley), water, boswellia gum, nigella sativa seeds, wheat and barley dispersion can be achieved by means of a high shear mixer such as a Silverson, which is well known to those skilled in the art. Other forms of colloid mill, fluid energy mill or high pressure homogenization may also be suitable. Conventional techniques such as wet milling, spray-drying and freeze-drying may also be used as part of the process. Additional ingredients of the formulation may be added either before or after homogenizations.

High pressure homogenization is an entirely mechanical process, in which the product is forced by a high pressure piston pump through a homogenizing valve. The commercially available Niro Soavi homogeniser is an example of a suitable high pressure homogenizer.

In the process of homogenization, however performed, the dimensions of the suspended particles of Petroselinum Crispum (parsley), water, boswellia gum, nigella sativa seeds, wheat and barley materials are reduced. At the end of the process, the suspension commonly presents a uniform distribution, according to a “Gaussian” curve, although such a particle size distribution may not be essential. The particle size distribution will vary with the operating conditions. The temperature at which Petroselinum Crispum (parsley), boswellia gum, nigella sativa seeds, wheat and barley materials are dispersed in water prior to or after homogenization and the homogenization pressure may require careful adjustment to achieve a commercially viable, stable, safe and elegant dispersion.

The yield and quality of the dispersion prepared by homogenization will depend on the variety, ripeness and dimensions of the components as well as on the degree of integrity of the herbs, seeds, fruits and vegetables used, the manner in which they have been stored, and the preparation technique.

The homogenate may include one or more additional nutritional components such as fats, carbohydrates, proteins, vitamins, drugs and minerals.

Fats or lipids that may be incorporated into the liquid formulation include, but are not limited to, coconut oil, soy oil, corn oil, olive oil, safflower oil, high oleic safflower oil, MCT oil (medium chain triglycerides), sunflower oil, high oleic sunflower oil, structured triglycerides, palm and palm kernel oils, palm olein, canola oil, marine oils, cottonseed oil and combinations thereof. Carbohydrates that may be incorporated into the formulation may be simple or complex, lactose-containing or lactose-free, or combinations thereof. Non-limiting examples of suitable carbohydrates include hydrolysed corn starch, maltodexthn, glucose polymers, sucrose, corn syrup, corn syrup solids, rice-derived carbohydrate, glucose, fructose, lactose, high fructose corn syrup and indigestible oligosaccharides such as fructooligosaccharides, and combinations thereof.

The homogenate may further comprise any of a variety of vitamins, non-limiting examples of which include vitamin A, vitamin B, vitamin B2, vitamin C, niacin, pyridoxine, vitamin E, folic acid, biotin, vitamin K, choline, inositol, salts and derivatives thereof, and combinations thereof.

The homogenate may further comprise any of a variety of electrolytes, non-limiting examples of which include catalytic elements, mineral salts, calcium, magnesium, potassium, sulfur, chlorine, arsenic, silicon, manganese, zinc, iodide, copper, and combinations thereof.

Another embodiment of the invention includes a liquid dosage formulation comprising one or more pharmaceutically active agents, wherein the formulation further comprises processed Petroselinum Crispum (parsley), boswellia gum, nigella sativa seeds, water, wheat and barley.

Taste-masking may be obtained by including additional viscosity enhancing agents, flavors and/or sweeteners. Flavoring agents that may be used in the present disclosure include, but are not limited to, natural flavors, natural fruit flavors, artificial flavors, artificial fruit flavors, flavor enhancers or mixtures thereof. The natural flavor may be selected from apple essence, pear essence, peach essence, berry essence, wildberry essence, blueberry essence, kiwi essence, strawberry essence, raspberry essence, cherry essence, plum essence, pineapple essence, and apricot essence, natural mixed berry flavor, citric acid, malic acid, vanilla, vanillin, cocoa, chocolate, and menthol. In addition, the flavors may be selected from one or more of the group consisting of anise oil, cinnamon oil, peppermint oil, oil of wintergreen, clove oil, bay oil, anise oil, eucalyptus oil, thyme oil, cedar leave oil, oil of nutmeg, oil of sage, oil of bitter almonds, cassia oil, lemon oil, orange oil, lime oil, grapefruit oil, grape oil and combinations thereof.

Natural flavors, artificial flavors or mixtures thereof include mint (such as peppermint or spearmint), menthol, cinnamon, vanilla, artificial vanilla, chocolate, artificial chocolate or bubblegum. Natural fruit flavors, artificial fruit flavors or mixtures thereof include, but are not limited to, cherry, grape, orange, strawberry or lemon. Flavour enhancers include, but are not limited to, citric acid. Although flavoring agents are generally provided as a minor component of the formulation, the addition of at least one flavoring agent is preferred. However, up to two flavoring agents may generally be employed.

The homogenate including taste masking components may further comprise an effective amount of a sweetener, at least one flavoring agent, and an artificial sweetening agent.

Optional sweetening agents include, but are not limited to, sugar sweeteners such as monosaccharides, disaccharides and polysaccharides. Examples of suitable sugar sweeteners include, but are not limited to, xylose, ribose, glucose, mannose, galactose, fructose, dextrose, sucrose, maltose, partially hydrolysed starch (such as maltitol syrup) or corn syrup, and sugar alcohols such as sorbitol, xylitol, mannitol, glycerin and combinations thereof. Artificial sweeteners include, but are not limited to, aspartame, acesulfame potassium, cyclamate, saccharin, saccharin sodium, sucralose and mixtures thereof.

The amount of additional sugars, optional and artificial sweetener used in the formulation will vary depending on the degree of sweetness and palatability desired.

The homogenate according to the disclosure are preferably formulated with a certain degree of viscosity, which may be imparted by the Petroselinum Crispum (parsley), boswellia gum, nigella sativa seeds, water, wheat and barley materials. Alternatively, additional viscosity enhancing agents may be included to achieve the optimum viscosity, taste masking and desired physical and/or chemical characteristics throughout product shelf life.

Examples for viscosity agents are poly(vinylpyrrolidone); polyvinyl alcohol; methylcellulose, hydroxypropyl methylcellulose, sodium carboxymethylcellulose, and mixtures thereof; sodium alginate; polyacrylamides; polyacrylic acids; collagen; polyethylene glycol; polysaccharides and carbohydrates such as starch, cellulose, dextrans and derivatives; thixotropic media; and the like. Other viscosity agents include natural gums such tragacanth, acacia or xanthan gum, guar gum, and gelatin, as well as clays such as veegum, bentonite, and hectorite.

In another embodiment the homogenate comprises of a preservative system that is used as an oral liquid such as the liquid formulations of the disclosure, and this may be selected from those conventionally employed in oral medicines. These usually consist of benzoic acid, sodium benzoate, potassium sorbate, ascorbic acid, sorbic acid, domiphen or other suitable preservatives and their mixtures.

As the homogenate of the disclosure may be thermodynamically unstable dispersed systems, in which the solid particles of the internal phase tend to aggregate and form sediment, the use of a coadjuvant may be required to improve dispersion, viscosity and other aspects so that a stable product is obtained.

An effective quantity of a wetting agent may be required if relatively insoluble drugs or ingredients are present in the composition. Such wetting agents may be selected from the group consisting of surface active agents, (e.g. anionic, non-ionic and cationic surfactants), glycerol, propylene glycol, liquid polyethylene glycols, sorbitol and mixtures thereof.

The homogenate may also contain demulcents additional to the Petroselinum Crispum (parsley), boswellia gum, nigella sativa seeds, wheat and barley materials, such as liquorice (Glycyrrhiza), to further enhance the demulcent properties of the formulation.

The following examples provide detailed illustrations of the compositions and methods of the present disclosure. These examples are not intended, however, to limit or restrict the scope of the invention in any way, and should not be construed as providing methods, conditions, ingredients or starting materials which must be utilized exclusively to practice the present disclosure.

### EXAMPLES

The homogenate was prepared and administered to 92 diabetic patients aged between 45-87 years old. 50 patients were diagnosed with type 1 diabetes and 42 patients with type 2 diabetes, and below are the clinical data that reflects the effectiveness of the previously described homogenate.

| | Glucose level | Glucose level | Glucose level |
|---|---|---|---|
| | (mg/dl) before | (mg/dl) during | (mg/dl) after |
| Patient | treatment | treatment | treatment |
| 1 | 420 | 311↓ | 110 |
| 2 | 370 | 290↓ | 120 |
| 3 | 392 | 288↓ | 119 |
| 4 | 411 | 354↓ | 121 |
| 5 | 370 | 290↓ | 120 |
| 6 | 399 | 277↓ | 118 |
| 7 | 434 | 322↓ | 125 |
| 8 | 389 | 279↓ | 122 |
| 9 | 372 | 275↓ | 117 |
| 10 | 299 | 222↓ | 120 |
| 11 | 318 | 265↓ | 111 |
| 12 | 367 | 259↓ | 123 |
| 13 | 371 | 292↓ | 95 |
| 14 | 410 | 301↓ | 101 |
| 15 | 366 | 210↓ | 111 |
| 16 | 388 | 201↓ | 98 |
| 17 | 423 | 297↓ | 127 |
| 18 | 333 | 301↓ | 117 |
| 19 | 365 | 269↓ | 100 |
| 20 | 364 | 293↓ | 94 |
| 21 | 345 | 261↓ | 121 |
| 22 | 233 | 227↓ | 120 |
| 23 | 376 | 291↓ | 110 |
| 24 | 388 | 200↓ | 125 |
| 25 | 400 | 232↓ | 122 |
| 26 | 333 | 211↓ | 101 |
| 27 | 299 | 201↓ | 90 |
| 28 | 311 | 273↓ | 103 |
| 29 | 321 | 275↓ | 113 |
| 30 | 425 | 225↓ | 108 |
| 31 | 369 | 261↓ | 110 |
| 32 | 339 | 271↓ | 107 |
| 33 | 360 | 250↓ | 104 |
| 34 | 432 | 300↓ | 102 |
| 35 | 378 | 265↓ | 109 |
| 36 | 412 | 322↓ | 121 |
| 37 | 456 | 322↓ | 120 |
| 38 | 345 | 270↓ | 100 |
| 39 | 377 | 299↓ | 101 |
| 40 | 333 | 224↓ | 97 |
| 41 | 358 | 214↓ | 99 |
| 42 | 346 | 248↓ | 94 |
| 43 | 433 | 342↓ | 117 |
| 44 | 338 | 270↓ | 109 |
| 45 | 334 | 248↓ | 98 |
| 46 | 379 | 258↓ | 93 |
| 47 | 364 | 259↓ | 94 |
| 48 | 368 | 265↓ | 95 |
| 49 | 423 | 227↓ | 91 |
| 50 | 332 | 200↓ | 110 |
| 51 | 336 | 253↓ | 98 |
| 52 | 339 | 271↓ | 92 |
| 53 | 389 | 229↓ | 97 |
| 54 | 378 | 237↓ | 90 |
| 55 | 354 | 199↓ | 102 |
| 56 | 401 | 372↓ | 122 |
| 67 | 373 | 255↓ | 94 |
| 58 | 388 | 283↓ | 91 |
| 59 | 421 | 395↓ | 119 |
| 60 | 372 | 222↓ | 109 |
| 61 | 342 | 213↓ | 99 |
| 62 | 335 | 205↓ | 118 |
| 63 | 368 | 248↓ | 112 |
| 64 | 349 | 223↓ | 94 |
| 65 | 352 | 221↓ | 93 |
| 66 | 399 | 268↓ | 99 |
| 67 | 403 | 229↓ | 98 |
| 68 | 339 | 291↓ | 91 |
| 69 | 349 | 283↓ | 110 |
| 70 | 338 | 242↓ | 88 |
| 71 | 406 | 312↓ | 98 |
| 72 | 389 | 286↓ | 97 |
| 73 | 337 | 285↓ | 94 |
| 74 | 409 | 339↓ | 114 |
| 75 | 391 | 290↓ | 123 |
| 76 | 409 | 322↓ | 115 |
| 77 | 379 | 285↓ | 95 |
| 78 | 386 | 275↓ | 97 |
| 79 | 398 | 297↓ | 91 |
| 80 | 349 | 269↓ | 93 |
| 81 | 408 | 306↓ | 115 |
| 82 | 369 | 276↓ | 97 |
| 83 | 377 | 285↓ | 95 |
| 84 | 324 | 212↓ | 121 |
| 85 | 456 | 246↓ | 97 |
| 86 | 375 | 231↓ | 111 |
| 87 | 379 | 239↓ | 98 |
| 88 | 339 | 269↓ | 92 |
| 89 | 367 | 256↓ | 90 |
| 90 | 378 | 271↓ | 93 |
| 91 | 338 | 229↓ | 120 |
| 92 | 366 | 293↓ | 116 |

Data shown in the table above reflects 100% success. All 92 diabetic patients experienced a significant drop (72-80% drop) in glucose level in the blood with the administration of the homogenate of the current disclosure. The first 50 patients in the above table are patients diagnosed with type 1 diabetes mellitus, the rest were diagnosed with type 2 diabetes mellitus. The glucose level of the patients was measured in the early morning of day 4 of the treatment period before any administration.

It was noted that patients with worse conditions of both types of diabetes were affected by some other medical conditions that are associated with diabetes such as chronic wounds (gangrene) and eye damage (corneal damage). These patients took a longer treatment period, more towards the higher end of the range (30 consecutive days) but eventually had the same result.

Experiments were conducted on rats. Diabetes Mellitus was induced in rats using streptozotocin (an intra-venous injection of 60 mg/kg), glucose levels before and after administration of the homogenate were collected and displayed in the table below.

| | Tests | Tests | Tests | | |
|---|---|---|---|---|---|
| | Glucose | Glucose level | Glucose | | |
| | level (mg/dl) | (mg/dl) | level | | |
| | before | during | (mg/dl) after | Control | Control |
| Rats | treatment | treatment | treatment | positive | Negative |
| 1 | 467 | 300↓ | 100 | 431 | 109 |
| 2 | 444 | 316↓ | 102 | 388 | 100 |
| 3 | 399 | 254↓ | 108 | 299 | 112 |
| 4 | 389 | 306↓ | 118 | 389 | 110 |
| 5 | 406 | 299↓ | 116 | 401 | 98 |
| 6 | 442 | 218↓ | 99 | 369 | 89 |
| 7 | 410 | 309↓ | 92 | 396 | 120 |
| 8 | 432 | 300↓ | 121 | 376 | 115 |
| 9 | 378 | 211↓ | 109 | 401 | 107 |
| 10 | 301 | 232↓ | 115 | 394 | |

Thus, the foregoing discussion discloses and describes merely exemplary embodiments of the present disclosure. As will be understood by those skilled in the art, the present disclosure may be embodied in other specific forms without departing from the spirit or essential characteristics thereof. Accordingly, the disclosure of the present disclosure is intended to be illustrative, but not limiting of the scope of the disclosure, as well as other claims. The disclosure, including any readily discernible variants of the teachings herein, define, in part, the scope of the foregoing claim terminology such that no inventive subject matter is dedicated to the public.

1. A method for treating and/or inhibiting the symptoms of type 1 diabetes or type 2 diabetes, comprising:
administering a homogenized composition comprising Petroselinum Crispum (parsley), boswellia gum, nigella sativa seeds, water, wheat and barley to a human suffering from type 1 diabetes or type 2 diabetes, wherein the homogenized composition is administered in an amount effective to decrease the glucose level in the blood and stimulate insulin secretion in the human,
wherein the homogenized composition is administered in a daily amount of at least 7.6 g/kg of the homogenized composition/kg of the human.

2. The method of claim 1 further comprising:
homogenizing the components.

3. The method of claim 1, wherein the composition is administered in an amount effective to reduce the blood glucose level from 72 to 80% based on the blood glucose level of the human immediately before the administering and the blood glucose level of the human 1 hour after the administering.

4. The method of claim 1 wherein the homogenized composition is prepared by mixing amounts of water and Petroselinum Crispum (parsley) for time sufficient to homogenize the composition, then adding amounts the boswellia gum, nigella sativa seeds, water, wheat and barley.

5. The method of claim 1 wherein the composition is administered orally to the human in liquid form.

6. The method of claim 5 wherein the composition is administered to the patient in the liquid form and wherein the composition further comprises at least one selected from the group consisting of a syrup, an artificial sweetening agent, and a natural fruit flavoring agent.

7. The method of claim 1 wherein the human is administered the homogenized composition all at once.

8. The method of claim 1 wherein the human is administered the composition for a minimum period of 7 consecutive days and a maximum period of 30 consecutive days.
